(19) United States
(12) Patent Application Publication
(10) Pub. No.: US 2006/0271042 A1
(43) Pub. Date: Nov. 30, 2006

(54) Cutting and coagulating electrosurgical forceps having cam controlled jaw closure

(75) Inventors: Scott T. Latterell, Minneapolis, MN (US); Douglas S. Wahnschaffe, Otsego, MN (US); Scott R. Sanders, Longmont, CO (US); Nigel Mark Goble, Berkshire (GB); Colin Charles Owen Goble, South Oxfordshire (GB); Gary Whipple, Attleboro, MA (US); A. David Boccuti, Arlington, VA (US); Edward R. Danis, Uxbridge, MA (US); John C. Robertson, Brooklyn, CT (US)

Correspondence Address: NIKOLAI & MERSEREAU, P.A., 900 SECOND AVENUE SOUTH, SUITE 820, MINNEAPOLIS, MN, 55402, US

(73) Assignee: Gyrus Medical, Inc., Maple Grove, MN (US)

(21) Appl. No.: 11/138,960

(22) Filed: May 26, 2005

(51) Int. Cl. A61B 18/14

(52) U.S. Cl. 606/51; 606/52

(57) ABSTRACT

A bipolar electrosurgical forceps comprises an elongated tubular barrel having a proximal end, a distal end and a lumen extending between these two ends. A handle is provided at the proximal end of the barrel and includes an actuating member for opening and closing a pair of forceps jaws that are mounted at the distal end of the barrel. The forceps jaws include cam slots in a proximal head portion thereof. A coupling member extends between the actuating member on the handle and the pair of forceps jaws. The coupling member includes drive pins that cooperate with the cam slots whereby squeezing of the actuating mechanism first effects pivotal rotation of the pair of forceps jaws over a first range of motion of the actuating member and translation without rotation of the forceps jaws over a second range of motion of the actuating member.

### BACKGROUND OF THE INVENTION

I. Field of the Invention

The present invention relates generally to electrosurgical instruments, and more particularly to a bipolar electrosurgical device for coagulation and cutting of target tissue and specifically designed for use in the performance of percutaneous laparoscopic surgery or other endoscopic procedures.

II. Background of the Invention

For a number of years, the medical device industry, in cooperation with healthcare providers, has been developing methods and devices to permit surgical procedures to be performed in a less invasive manner. Minimally invasive surgery generally involves the use of instruments that avoid the need to make major incisions in the body. Major incisions usually require a relatively long period of hospitalization and subsequent home recovery. Minimally invasive surgery has the salutary effects of shortening hospital stays and recovery times.

Minimally invasive surgical procedures are generally performed through a trocar cannula. The cutting and coagulating instruments most often used are either electrosurgical or laser-based. While, generally speaking, laser based instruments are capable of more precise cutting than electrosurgical instruments, they are somewhat difficult to control, particularly in the close conditions of laparoscopic procedures.

Electrosurgical instruments are either monopolar or bipolar in nature. In monopolar electro surgery, there is a greater potential for injury to body tissues because an electric current most pass through the tissues on a “path of least resistance” basis to a return electrode located on the patient's skin. In laparoscopic procedures, there is even a greater potential for complications when using monopolar instruments, due to the combined effects of the surgeon's limited field of vision, the proximity of other organs to the tissue being cut and the inherent tendency of monopolar RF energy to find a somewhat random path back to the return electrode.

Bipolar electrosurgical instruments provide an improved margin of patient safety in certain minimally invasive surgical and interventional procedures. In the case of bipolar devices, the RF energy is contained at the surgical site because both the active electrode and the return electrode are located in close proximity to one another on the surgical instrument itself.

Bipolar coagulating and cutting forceps are known in the art, examples of which may be found in U.S. Pat. Nos. 5,258,006, 5,445,638, and 5,562,546 to Rydell et al. The forceps jaws are used for grasping or gripping the tissue to be cut. A RF current may be made to pass through the tissue disposed between the clamped jaws to desiccate the tissue and then, in the case of the Rydell et al. '638 patent, a mechanical cutting blade may be actuated and made to pass through the clamped tissue to sever it.

U.S. Pat. No. 5,735,849 to Baden et al. describes an endoscopic, electrosurgical forceps having an elongated tubular barrel that has coagulating electrodes on the opposed jaw surfaces and a cutting blade similar to that which is disclosed in the Rydell '638 patent but with an improved handle mechanism that allows the surgeon to select between a bi-directional mode or a uni-directional mode of jaw movement. In each instance, however, the electrode surfaces on the jaws approach one another in a relative parallel relationship during jaw closure and likewise, separate from one another in a relative parallel relation as the jaws are made to open. Maintaining this parallel relationship results in improved and more uniform coagulation but suffers a drawback that the amount of separation of the opposed jaw surfaces is somewhat limited, making it more difficult to place the open jaws about target tissue prior to jaw closure, desiccation and cutting.

Consequently, a need exists for an electrosurgical forceps instrument that allows significantly wider jaw separation when the jaws are fully opened relative to one another but which will assume a more parallel movement as they are made to close upon target tissue captured therebetween.

Given the constraints imposed on the surgeon while performing minimally invasive surgery through a trocar or the like, it would also be advantageous if the jaw assembly can be redirected via a control lever on the instrument's handle without having to reposition the trocar. The ability to redirect the angle at which the jaws extend from the instrument's tubular barrel facilitates the ability of the surgeon to gain purchase to target tissue during the course of a laparoscopic procedure. This is especially true when the instrument also has the capability of being able to rotate the barrel and jaw assembly without moving the handle, a feature disclosed in the Rydell et al. '006 patent.

### SUMMARY OF THE INVENTION

The above-described drawbacks of prior art electrosurgical forceps are obviated by the cutting and coagulating electrosurgical forceps of the present invention. It comprises an elongated tubular barrel having a proximal end, a distal end and a lumen extending therebetween. A handle is provided at the proximal end of the barrel and includes an actuating member on the handle. A pair of forceps jaws is mounted at the distal end of the barrel where each of the forceps jaws has cam slots formed through a proximal head portion to which the tissue engaging surfaces of the jaws are joined. A coupling member extends between the actuating member on the handle and the pair of forceps jaws where the coupling member includes members that cooperate with the cam slots such that by squeezing the actuating mechanism effects pivotal rotation of the pair of forceps jaws over a first range of motion of the actuating member and translation without rotation of the pair of forceps jaws over a second range of motion of the actuating member.

For example, and without limitation, the handle may have a pistol grip configuration with the “actuating member” being the trigger on the pistol grip. Initially, the individual jaw members may be at a predetermined angle with respect to one another and as the trigger is squeezed, the angle decreases to a point where the opposed jaw surfaces carrying the electrodes are parallel to one another and then continued squeezing of the trigger over a second range of motion brings the two jaws together in a parallel manner.

As a further feature of the invention, a thumb lever is provided on the handle that is operatively coupled to the jaw assembly which is pivotally secured at the distal end of the elongated barrel whereby actuation of the thumb lever redirects the angle at which the jaw assembly projects from the distal end of the elongated barrel.

The instrument further includes a reciprocally movable cutting blade disposed at the distal end of the tubular barrel and it can be made to project out the distal end of the barrel between the closed forceps jaws through actuation of a pushrod that is attached to the blade and that extends through the lumen of the barrel to a control lever on the handle.

### DESCRIPTION OF THE DRAWINGS

These and other objects, advantages and features of the invention will become apparent to persons skilled in the art from the following detailed description of a preferred embodiment depicting the best mode contemplated for carrying out the invention. In the drawings, like numerals in the several views refer to corresponding parts.

FIG. 1 is a side elevation view of the cutting/coagulating electrosurgical forceps comprising the preferred embodiment of the present invention;

FIG. 2 is a longitudinal cross-sectional view revealing the internal working parts of the instrument;

FIG. 3 is an enlarged detailed view of the distal end portion of the instrument, again in cross section;

FIG. 4 is an exploded view of the jaw assembly illustrating its manner of attachment to the distal end of the instrument's barrel.

FIG. 5 is a view illustrating the articulated mount of the jaw assembly to the instrument's barrel;

FIGS. 6A-6E illustrate the jaw member achieved by providing specially shaped cam grooves on the instrument's jaw members; and

FIG. 7 is an exploded view of one jaw member and its associated electrode structure.

### DESCRIPTION OF THE PREFERRED EMBODIMENT

Certain terminology will be used in the following description for convenience in reference only and will not be limiting. The words “upwardly”, “downwardly”, “rightwardly”, “leftwardly”, “distally” and “proximally” will refer to directions in the drawings to which reference is made. The words “inwardly” and “outwardly” will refer to directions toward and away from, respectively, the geometric center of the device and associated parts thereof. Said terminology will include the words above specifically mentioned, derivatives thereof and words of similar import.

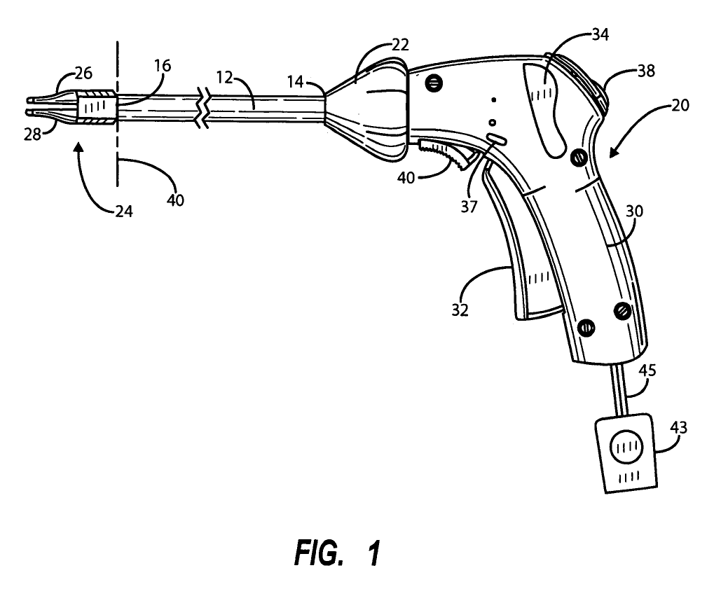

Referring to FIG. 1, there is indicated generally by numeral 10 a bipolar electrosurgical forceps comprising a preferred embodiment of the present invention.

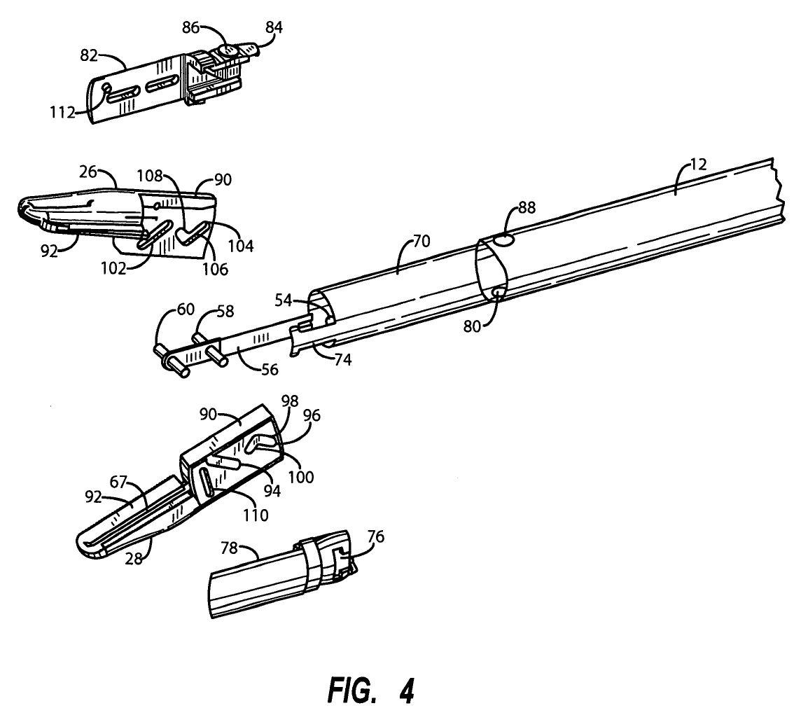

It is seen to include an elongated tubular barrel 12 having a proximal end 14, a distal end 16 and a lumen 18 (FIG. 3) extending therebetween. Disposed at the proximal end 14 of the tubular barrel is a handle assembly indicated generally by numeral 20. The handle assembly 20 includes a rotatable knob 22 to which the barrel 12 is secured such that rotation of the knob 22 will rotate the barrel 12 about its longitudinal axis. Internal stops are provided which only permits rotation of knob 22 and the barrel through an angle that may be in a range from about 180° to close to 360°.

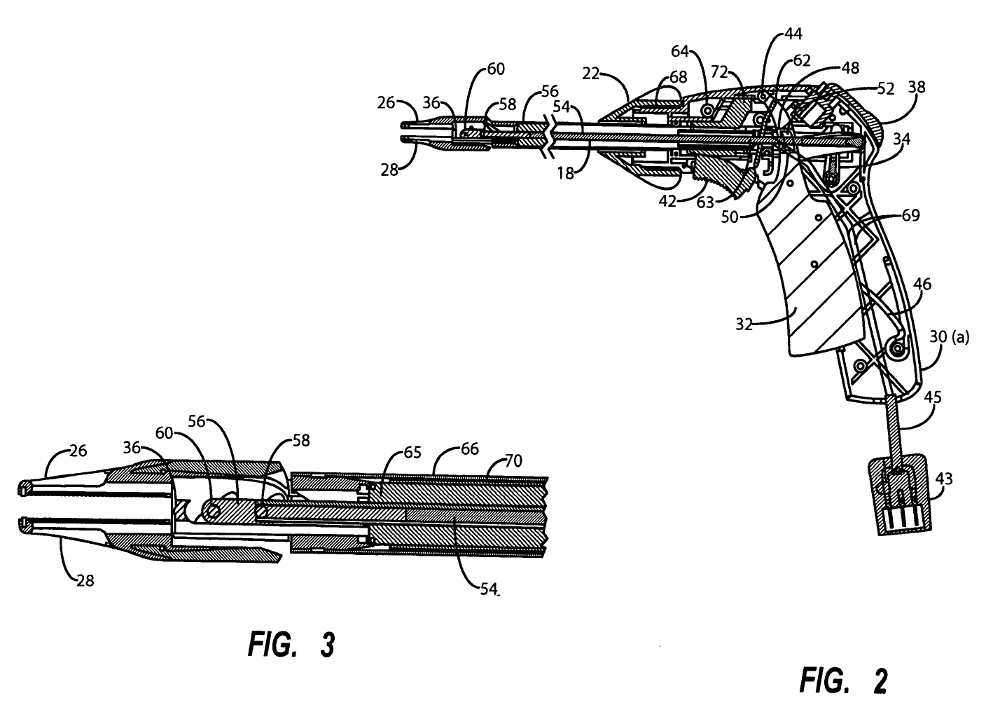

Located at the distal end 16 of the tubular barrel 12 is a forceps jaw assembly 24 including a pair of cooperating jaw members 26 and 28 (FIG. 2). As will be further explained herein below, the handle assembly 20 has a pistol like configuration with a handle member 30 adapted to be held in the palm of the surgeon's hand and with a trigger 32 extending outward through a slot in the handle. The trigger 32 is operatively coupled to the jaw assembly 24 such that squeezing the trigger 32 imparts movement of the jaw members 26 and 28 relative to one another. The mechanism for imparting the particular blade movement will be explained in greater detail when FIGS. 2 and 3 are described.

Projecting out from opposed side surfaces of the handle 30 are thumb levers 34 which when depressed so as to pivot counterclockwise when viewed in FIG. 1 cause a blade 36 (FIG. 2) to extend outward from the distal end of the instrument 10, traveling through slots formed through the mating jaw surfaces of jaw members 26 and 28. Ears, as at 37 in FIG. 1, project from the sides of the handle to limit the extent of counter-clockwise travel of the lever 34.

Also shown in FIG. 1 is a further thumb lever 38 that is pivotally mounted in the handle 30 and which operatively connects to the jaw assembly 24 to cause the jaw assembly 24 to articulate about an axis represented by dashed line 40 in FIG. 1.

Another feature illustrated in FIG. 1 is the presence of a ratchet release trigger 42 that projects out through a slot formed in the undersurface of the handle 30. Depression of this lever will release a ratchet-like mechanism yet to be described for maintaining a setting of the jaw members 26 and 28 relative to one another.

An electrical adapter 43 connects to a cord 45 that extends through the handle and down the tubular barrel's lumen 18 to connect to electrode structures on facing surfaces of jaws 26 and 28 in a manner to be further described herein below.

Having generally described the basic constructional features of the bipolar electrosurgical forceps, consideration will next be given to the mechanisms contained in the handle for controlling movements of the jaw assembly 24 from side to side, the movement of the jaws 26 and 28 from their open to their closed position, the movement of the instrument's cutting blade and the rotation of the blade assembly 24 through about 180° with respect to the longitudinal axis of the barrel 18.

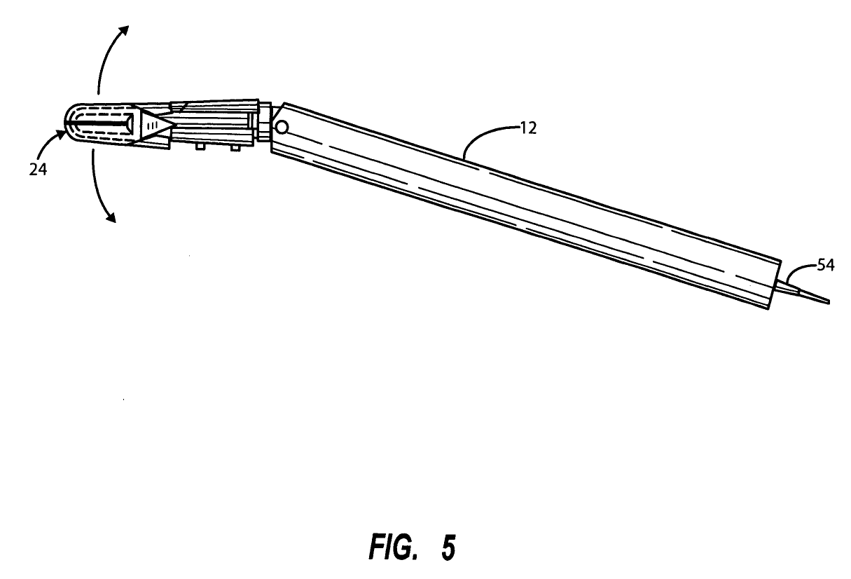

With continued reference to FIG. 2 which shows a longitudinal cross-sectional view taken down the midline of the instrument, the trigger member 32 pivotally connects to the handle half 30(a) by means of a trigger hinge pin 44. As in the aforereferenced Baden et al. U.S. Pat. No. 5,735,849, the trigger 32 is spring-biased by a spring wire 46 to project outward of the handle member 30. As the trigger 32 is squeezed, it compresses the spring wire 46. A bore 48 is formed through the trigger member 32 and fitted into this bore and held in place by a pin 50 is a trigger support 52 that is attached to a jaw drive rod 54. The jaw drive rod is, in turn, braised or otherwise attached proximate its distal end to a flat flexible steel strap 56 that supports two spaced-apart, transversely-extending drive pins 58 and 60 that are more clearly seen in the enlarged exploded view of FIG. 4.

The jaw drive rod 54 passes through a small aperture formed through a pawl 62 that is canted at a slight angle to the axis of the drive rod by a spring 63 and the tolerances are such that when the pawl is canted at an angle to the axis of the drive rod 54, the drive rod can be moved in the proximal direction as the trigger 32 is squeezed, but is prevented from returning in the distal direction by the frictional engagement between the rod 54 and the pawl 62. However, when the pawl 62 is oriented perpendicularly to the drive rod 54 rather than at an angle, there is no longer interference between the pawl and the drive rod and the spring 46 is able to move the drive rod 54 in the distal direction. Actuation of the ratchet release 42 serves to orient the pawl 62 perpendicularly to the drive rod.

Also extending through the lumen 18 of the tubular barrel 12 is a blade push tube 65 that is affixed to the thumb lever 34 and that surrounds the jaw drive rod 54.

The thumb lever 34 is urged in the clockwise direction when viewed as in FIG. 1 by means of a spring 64 such that thumb pressure must be brought to bear on the lever 34 in order to displace the blade 36 out the distal end of the tubular barrel and along the slots 67 (FIG. 4) provided in the opposed faces of the jaw members 26 and 28. These slots permit the blade member 36 to pass along the length of the jaw member as the lever 34 is manipulated to cut through desiccated tissue clamped between the opposed forceps jaws.

With continued reference to FIG. 2, plastic shrink tubing 66 surrounds the outer barrel 12. A knob 22 is rotatably supported on a cylindrical hub 68 on the distal end portion of the handle 30 with the knob being fixedly attached to the outer circumference of the shrink tubing covered barrel 12. Thus, rotation of the knob 22 also rotates the barrel and the jaw assembly secured to the distal end thereof. A boss on the inside of the spindle travels in a slot in the distal end of the handle. The slot only runs 90° each direction from the top center. Thus, the barrels i limited to about 180° of rotation. This prevents undue twisting of wires 69 in cord 45. This slot could be increased to give close to 360° of rotation, if desired.

Turning next to the exploded view of FIG. 4, it can be seen that an articulation drive tube 70 is concentrically disposed within the lumen of the outer barrel 12 and is reciprocally movable therein. The proximal end of the articulation drive tube is attached to a slide member 72 disposed within the handle 30 and is operatively coupled to the lever 38 that is rotatably mounted with respect to the upper rear portion of the handle as shown in FIGS. 1 and 2. Movement of the handle to the left or right imparts axial movement of the articulation drive tube 70.

Formed on the distal end of the articulation drive tube 70 is a T-shaped tab 74 that is designed to fit within a T-shaped pocket 76 formed on a jaw support member 78. While not visible in FIG. 4, the jaw support member 78 has a cylindrical protuberance on an undersurface thereof that is adapted to fit into a circular aperture 80 formed proximate the distal end of the outer tube 12. Likewise, a second jaw support member 82 has a hub portion 84 from which a further cylindrical protuberance 86 projects. The protuberance 86 is adapted to fit within an aperture 88 in the distal end portion of the outer tube 12. It is to be noted that the tab 74 on the articulation drive tube 70 is laterally offset from the axis defined by the aligned apertures 80 and 88 and, thus, when the articulation drive tube 70 is reciprocally moved, the jaw support members 78 and 82 will be made to pivot about that axis.

Sandwiched between the jaw supports 78 and 82 are the forceps jaw members 26 and 28. Each jaw member includes a head portion 90 and a jaw portion 92. The head portion 90 of the jaw member 28 includes a first slot 94 that is diagonally oriented relative to a longitudinal axis of the jaw member 28. A second cam slot 96 is also formed on the head portion 90 of the jaw member 28 and it is somewhat L-shaped with a stem segment 98 of the L generally aligned parallel to the cam slot 94 and a base segment 100 of the L that is at a predetermined angle to the stem segment 98. Likewise, the head portion 90 of the jaw member 26 has a cam slot 102 oriented diagonally to a longitudinal axis of jaw member 26 as well as a complimentary L-shaped cam slot 104 with a stem segment 106 that runs parallel to the cam slot 102 and a base segment 108 that is at an angle to the stem segment.

The hub 90 of the jaw member 28 further includes a slot 110 that is oriented generally perpendicular to the longitudinal axis of the jaw member. While not visible in FIG. 4, the head portion of jaw member 26 also has a slot-like that of slot 110 on jaw member 28.

When the jaw supports 78 and 82 are made to sandwich the head portions jaw members 26 and 28, the drive pins 58 and 60 projecting laterally from the drive strap 56, fit into the above-described cam slots on the jaw members. More particularly, drive pin 58 is made to reside in the cam slots 96 and 104 while drive pin 60 extends into the slots 94 and 102. Short, stub-like pins, as at 112, project outward from the inner face of the jaw support members so as to reside in the slots 110 in the respective jaw members.

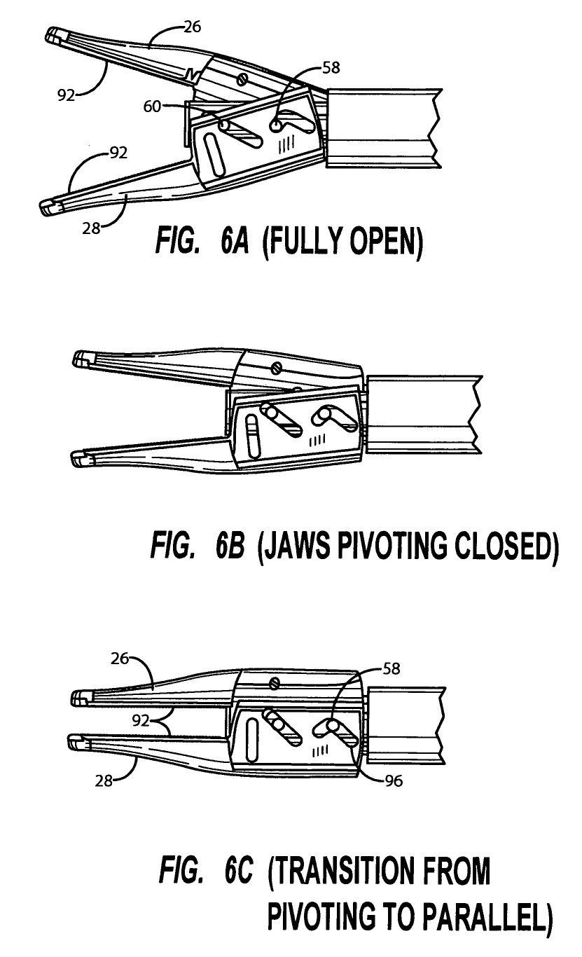

With the jaw supports and jaws so arranged and with the protuberances, like 86 on the jaw support members 82 and 78, located within the apertures 80 and 88, the jaw assemblies can be made to swing through a predetermined arc, as illustrated in FIG. 5, when the thumb lever 38 is pushed from one side to the other on the handle 30.

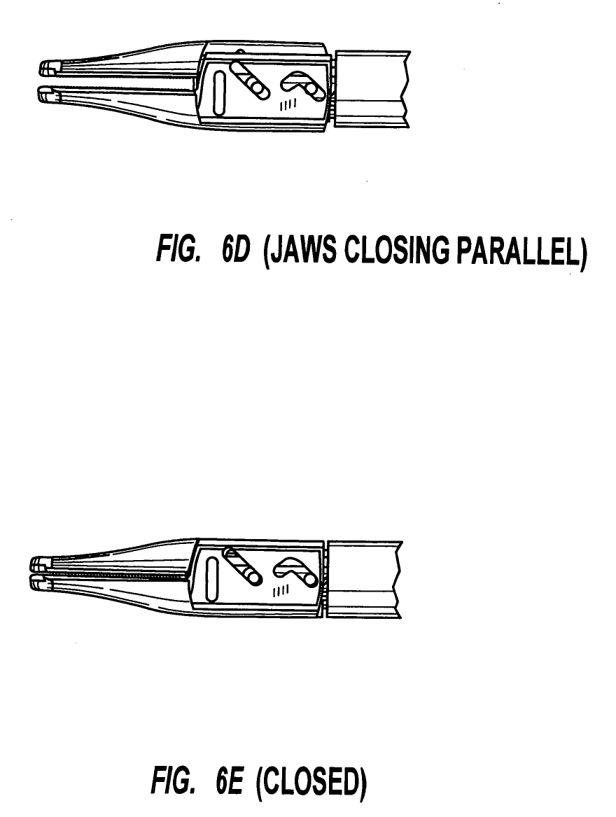

Turning now to FIG. 6A when the drive pins 58 and 60 are all the way forward in the distal direction of the instrument, as they will be when trigger 32 is not being squeezed, the opposing jaw surfaces 92 of the blade members 26 and 28 are at an angle to one another, providing the maximum opening between the distal tips of the two jaws. As the trigger 32 is begun to be squeezed and the drive pins 58 and 60 are pulled rearward, the caming action between the drive pins and the slots in which they reside causes the distance between the jaw tips, as well as the angle between the opposed jaw surfaces 92, to decrease. With reference to FIG. 6C, as the surgeon continues to squeeze the instrument's trigger 32 over a first range of motion, the drive pin 58 reaches the point where it exits the base of the L-shaped cam slots 96 and 104 and enters the stem segment thereof. At this point, the jaw surfaces 92 on the jaws 26 and 28 assume a parallel relationship to one another. Continued squeezing of the instrument's trigger over a second range of motion causes the jaw surfaces 92 to close together, moving in a parallel relationship to one another. See FIGS. 6D and 6E.

It is to be seen, then, that by providing the cam slots in the jaw member heads, the resulting electrosurgical forceps instrument allows significantly wider jaw tip separation when the jaws are fully open relative to one another, but that will assume a parallel movement as they are made to close upon target tissue captured between the tissue engaging portions of the jaws. This results in greater ease in gaining purchase of the target tissue while still providing improved electrocoagulation achieved when parallel jaw movement is involved.

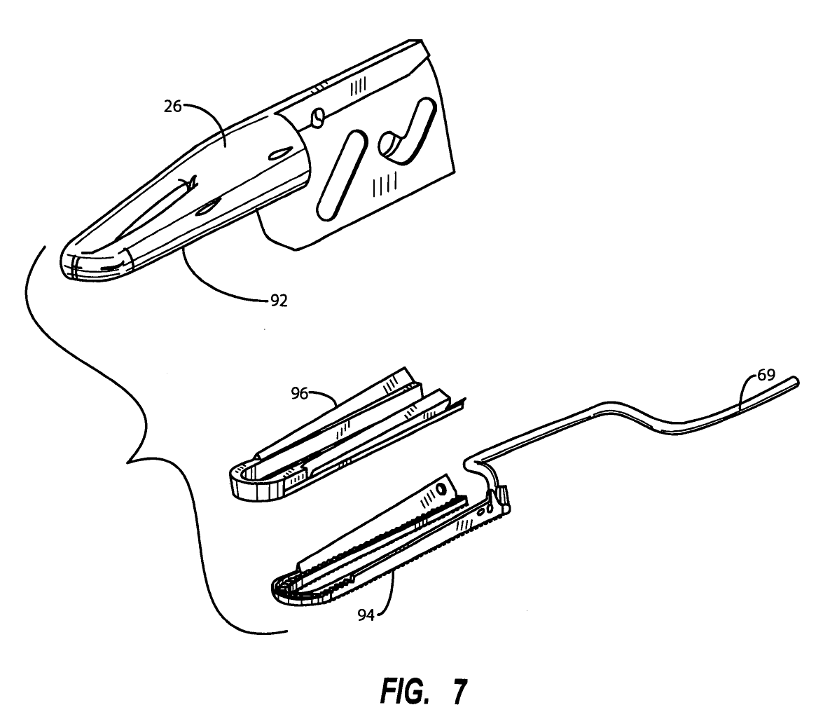

FIG. 7 is an exploded view of the jaw member 26 showing the manner in which an electrode is mounted on the jaw in opposing relation to a similar electrode mounted on the opposite jaw 28. The jaw member 26 is fabricated from metal and to insulate the jaw member body from the electrode 94, an insulating shim 96 is bonded to the jaw surface 92 and the electrode, in turn, is bonded to the insulating layer so as to be in a non-contact relationship with the metal jaw member 26. The wire 69 that leads to the electrical plug or connector 43 attaches to the electrode 94.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. A cutting and coagulating electrosurgical forceps comprising:
(a) an elongated tubular barrel having a proximal end, a distal end and lumen extending therebetween;
(b) a handle with an actuating member affixed to the proximal end of the barrel;
(c) a pair of forceps jaws mounted at the distal end of the barrel; and
(d) a coupling member extending between the actuating member and the pair of forceps jaws whereby movement of the actuating member effects primarily pivotal movement of the pair of forceps jaws over a first range of motion of the actuating member and primarily translational movement of the pair of forceps jaws over a second range of motion of the actuating member.

2. The cutting and coagulating electrosurgical forceps as in claim 1 wherein the tubular barrel is rotatable with respect to the handle.

3. The cutting and coagulating electrosurgical forceps as in claim 2 wherein the tubular barrel is rotatable with respect to the handle through a predetermined angle in a range of from about 180° to about 360°.

4. The cutting and coagulating electrosurgical forceps as in claim 1 and further including a reciprocally movable cutting blade disposed at the distal end of the tubular barrel with a push rod attached to said blade and extending through the lumen to a control lever on the handle.

5. The cutting and coagulating electrosurgical forceps as in claim 1 wherein the pair of jaws is supported by a hub member at the distal end of the tubular barrel, the hub member being swivelable with respect to the distal end of the tubular barrel.

6. The cutting and coagulating electrosurgical forceps as in claim 1 wherein the pair of forceps jaws each include a distal portion with a tissue engaging surface integrally joined to a proximal head portion, the tissue engaging portion including electrode surfaces thereon and the head portions including first and second cam surfaces formed thereon.

7. The cutting and coagulating electrosurgical forceps as in claim 6 wherein the first cam surface comprises a rectilinear slot oriented oblique to a longitudinal axis of the forceps jaws and the second cam surface is generally L-shaped with a first segment aligned parallel to said rectilinear slot and a second segment generally perpendicular to the first segment.

8. The cutting and coagulating electrosurgical forceps as in claim 7 wherein the coupling member comprises an elongated generally rigid, reciprocally movable strip, the strip supporting a pair of drive pins that are spaced from one another and sized to cooperate with the slots comprising the first and second cam surfaces formed on the proximal head portions of the pair of forceps jaws.

9. The cutting and coagulating electrosurgical forceps as in claim 6 wherein the tissue engaging surface of the jaws include a medial slot, said forceps further including a reciprocally movable cutting blade disposed at the distal end of the tubular barrel with a push rod attached to said blade and extending through the lumen to a control lever on the handle, the cutting blade moving through the medial slots.

10. The cutting and coagulating electrosurgical forceps as in claim 6 and further including means for applying an RF voltage between the electrode surfaces on the pair of forceps jaws.

11. The cutting and coagulating electrosurgical forceps as in claim 5 wherein the handle further includes a thumb lever thereon, the thumb lever being operatively coupled to the hub member for swiveling the pair of jaws.

12. The cutting and coagulating electrosurgical forceps of claim 8 wherein the handle comprises a pistol grip and the actuating member comprises a trigger lever coupled to the rigid, reciprocally movable strip.

13. The cutting and coagulating electrosurgical forceps of claim 1 wherein the handle comprises a pistol grip and the actuating member comprises a trigger lever coupled to said coupling member.

14. The cutting and coagulating electrosurgical forceps of claim 7 and further including a pair of jaw support members disposed in covering relation to the proximal head portions of the pair of forceps jaws.

15. The cutting and coagulating forceps of claim 14 wherein the proximal head portion of each of the pair of forceps jaws includes a further slot oriented perpendicular to the longitudinal axis of the forceps jaws and each of the pair of jaw support members includes a pin projecting normal to an inner surface slot in the proximal head portion of the forceps jaw that is directly adjacent the jaw support members.

16. The cutting and coagulating electrosurgical forceps of claim 14 wherein the jaw support members include a proximal hub portion that pivotally joins to the distal end portion of the tubular barrel.

17. The cutting and coagulating electrosurgical forceps as in claim 1 wherein the coupling member is operative to effect only pivotal movement of the pair of forceps jaws over a first range of motion of the actuating member and only translational movement of the pair of forceps jaws over a second range of motion of the activating member.
